(19) United States
(12) Patent Application Publication
(10) Pub. No.: US 2022/0339087 A1
(43) Pub. Date: Oct. 27, 2022

(54) Acidic Stick Deodorants

(71) Applicant: Lume Deodorant, LLC, Chaska, MN (US)

(72) Inventors: Jeffrey Dean Lindsay, Appleton, WI (US); Shannon E. Klingman, Chaska, MN (US)

(21) Appl. No.: 17/486,373

(22) Filed: Sep. 27, 2021

Related U.S. Application Data
(63) Continuation of application No. 17/238,102, filed on Apr. 22, 2021.

(51) Int. Cl. A61K 8/44; A61K 8/67; A61K 8/92; A61K 8/36; A61K 8/34; A61K 8/891; A61K 8/73; A61K 8/24; A61K 8/41; A61K 8/46; A61Q 11/00; A61Q 15/00

(52) U.S. Cl. CPC A61K 8/447; A61K 8/673; A61K 8/922; A61K 8/361; A61K 8/345; A61K 8/891; A61K 8/732; A61K 8/24; A61K 8/416; A61K 8/466; A61Q 11/00; A61Q 15/00; A61K 8/676; A61K 2800/874; A61K 2800/30; A61K 2800/805

(57) ABSTRACT

Products and methods are disclosed relating to a deodorant stick comprising mandelic acid.

### BACKGROUND

### Field of the Invention

This invention pertains to products and methods for personal care, particularly personal care compositions and methods of use for reducing or preventing unwanted perspiration or odor associated with perspiration.

### Description of Related Art

Many people seek to avoid the embarrassment or discomfort associated with perspiration or associated odors. Bacteria such as Corynebacteria feed off materials in the sweat, particularly the apocrine sweat glands found under the arms and elsewhere, and produce unpleasant odors. Many people purchase antiperspirants or deodorants for underarm use, for example, to mask odors or reduce perspiration. Rather than relying on fragrance to mask odor or on metal-containing compounds such as aluminum and zirconium salts to plug up sweat pores, novel approaches have been developed that can reduce odor by adjusting the skin microbiome, the array of microbes that live on the skin. Two patents of Shannon Klingman, U.S. Pat. No. 9,566,223, “Antiperspirants and Deodorants,” issued Feb. 14, 2017, and U.S. Pat. No. 8,992,898, “Antiperspirants and Deodorants,” issued Mar. 31, 2015, both of which are hereby incorporated by reference, describe the use of alpha hydroxy acids, particularly mandelic acid, in combination with caffeine for control of the skin microbiome as a natural deodorant that can dramatically reduce odor without requiring the use of aluminum or zirconium salts, and a third from the same inventor, U.S. Pat. No. 9,668,948, “Products and Methods for Reducing Malodor from the Pudendum,” issued Jun. 6, 2017, hereby incorporated by reference, describes compositions with mandelic acid for controlling odor from the pudendum. A related line of deodorant products marketed by Lume Deodorant, LLC of Chaska, Minneapolis (lumedeodorant.com) have captured a significant portion of the natural deodorant market with its oil/water emulsion products containing mandelic acid. Unfortunately, conventional formulations suitable for natural deodorants in stick form faced significant challenges since the low pH range desired for the deodorant product and the amount of free mandelic acid required would either cause instability in the preparation due to incompatibility of many emulsifiers and others agents with low pH (e.g., from about 3 to 4.5 or from 3 to 4 or from 3.2 to 3.9, etc.), or result in skin irritation with encapsulated or isolated mandelic acid in a heterogeneous mixture since pockets of elevated acid content could cause small regions of irritated skin. Early efforts to make such an acidic stick in the bases used for other aluminum-free deodorants resulted in problems such as instability of a bend or the precipitation of acidic solids as rough grains or crystals in what was intended to be a smooth deodorant composition. Fortunately, Applicants have now developed a novel method and formulation approach that has overcome previously recognized problems, allowing a novel product to provide effective amounts of mandelic acid in a smooth, easy-to-use, substantially uniform deodorant stick.

### SUMMARY

We have found that personal care deodorant and antiperspirant compositions comprising effective levels of alpha hydroxy acids such as mandelic acid can be formulated in a solid stick for convenient application to the underarms or other regions of the body. Such sticks can also comprise caffeine and may be substantially aluminum free and zirconium free. In some embodiments, the composition is prepared by combining mandelic acid in a solvent with a thickener such as a starch, a gum, minerals such as laponite, other agents that swell in water or other solvents, or combinations thereof. The resulting thickened acidic mixture can then be combined with an oil phase to form an emulsion or other mixture of mandelic acid with the oil phase. For example, we have found that an aqueous solution of mandelic acid can be used, such as a solution comprising from 2% to 40% mandelic acid in water at a suitable temperature, or water combined with other solvents such as ethanol, 1-2 propanediol, 1-3 propanediol, or other alcohols, glycols, or esters. In some cases, the solution may be supersaturated or substantially saturated. The mandelic acid solution can then be combined with a thickener such as corn starch, tapioca starch, potato starch, cassava, arrowroot starch, chemically modified starches such as modified food starch and the like, with starch levels relative to the solvent mass of 0.5 to 15 weight percent, such as at least 0.5, 1, 2, 3, 4, 5, or 6 weight percent, up to one of any suitable integer from 2 to 15 weight percent, from 2 to 10 wt %, from 2 to 6 wt %, etc. When starch is used, the starch and solvent, which may be water or an aqueous solvent, is then heated until the starch grains swell and cause the slurry to become a thickened paste. In water-starch slurries, this may occur between 50° C. and 85° C., for example, with many native starches tending to gel around 55° C. to 65° C. Rather than gelling with the addition of heat, a soluble starch may be used that is soluble in cold water. The resulting acidic starch paste has elevated viscosity and reduced opacity relative to the initial slurry. An appropriate amount of this slurry, which may be heated to temperature from 40° C. or 50° C. to 70° C. or 80° C., for example, and can then be combined with a molten waxy phase to create a dispersion or emulsion that does not readily separate. Emulsifying waxes may be present but need not be used in many embodiments. The dispersion may be continually stirred or otherwise blended using rotary mixers, whisks, homogenizers, static mixers, etc., and may be kept at an elevated temperature for a suitable time to promote evaporation of the solvent or especially a portion of the water. The dispersion may then be blended with additional agents such as powdered starch or other powders including laponite, talc, hydroxyapatite, magnesium hydroxide, magnesium stearate, zinc stearate, zinc oxide, antiperspirant salts such as aluminum and zirconium salts, silica, microspheres, and the like.

In another embodiment, a water/oil emulsion or dispersion comprising mandelic acid associated with an aqueous solvent in an aqueous phase is heated to drive off a portion of the water such as at least 20%, at least 40%, or at least 60% of the water, with exemplary ranges of 20% to 90% or 30% to 80%, resulting in a highly uniform distribution of mandelic acid throughout a waxy phase. Continued stirring may applied during heating as water is driven off. The peak temperature of the mixture in this process may be at least 75° C., 80° C., 90° C., 95° C., 99° C., 100° C., 110° C., 115°, or in general any whole number between 75° C. and 150° C., such as from 85° C. to 140° C. or 85° C. to 130° C. or from 90° C. to 125° C. The composition may remain above 75° C., 80° C., 85° C., 90° C., 95° C., 99° C., 100° C. or 110° C. for at least 1 minute or any whole number of minutes from 1 to 90, such as from 1 minute to 30 minutes or for at least 2 minutes.

For example, mandelic acid may be dissolved in a mixture of water and an alcohol such as ethanol that may also comprise other soluble material or other solvents such as glycerin or propanediol, and this ethanol-water-acid mixture is combined with a mix of waxes, fatty acids, esters, butter, oils, and related lipids, optionally including at least one emulsifying wax. If no thickening agent is included, emulsifying waxes can be particularly helpful in promoting good mixing of the aqueous phase with the oil phase. As the combination is heated and stirred, the solids melt and begin to form a dispersion or emulsion with the ethanol-water-acid mix dispersed in the oil phase. As heating continues, water and ethanol may be progressively driven off, resulting in a waxy material with mandelic acid finely dispersed throughout. Prior to cooling, the melt may be combined with a starch such as arrowroot or tapioca starch to provide additional body and tactile properties, and may then be poured into a suitable container such as an oval-shaped deodorant container or mold.

Thus, in one embodiment, we have developed a personal care composition in the form of a deodorant stick for reducing at least one of perspiration and body odor, the stick comprising at least 0.2% by weight of mandelic acid distributed substantially uniformly throughout a solid or semi-solid waxy phase, such that the composition at room temperature is free of tangible or visible mandelic acid grains or crystals in a suitable carrier for application to the skin. In some embodiments, the mandelic acid is associated with starch granules dispersed throughout the stick that may be swollen, never swollen, or swollen and at least partially dried after contact with the mandelic acid. In some embodiments, the stick comprises two forms of starch, one that was previously gelatinized (swollen) in the presence of mandelic acid, and one that was added as unswollen particles to a molten waxy mix comprising previously the swollen starch particles associated with mandelic acid. Both the swollen and unswollen starch particles may independently comprise one or more starches such as corn starch, tapioca starch, and arrowroot starch.

Caffeine or other xanthines may be present and may be applied by fully or partially dissolving caffeine in an oil phase prior to combination with mandelic acid materials, or may be fully or partially dissolved in the mandelic acid solution or other aqueous phase to be combined with the oil phase. We have obtained surprisingly good results by adding caffeine powder to the oil phase where a portion appears to dissolve, and then combining a mandelic acid mixture such as mandelic acid in a starch paste, a gum-based paste or gel, etc., wherein the caffeine appears to be dissolved or finely dispersed in the heated mixture. Without wishing to be bound by theory, the caffeine is believed to provide beneficial functions by serving as a vasoconstrictor that can help close pores to reduce sweating, but also by a protective or anti-irritant function that reduces the risk of skin irritation from the presence of mandelic acid on the skin. Caffeine may be present in the oil phase at a level of 0.1 to 7%, from 0.3% to 5%, from 0.5% to 3%, and from 0.3% to 2%. Relative to the final composition, the caffeine concentration may be from 0.05% to 5%, from 0.1% to 4%, and from 0.3% to 3%. In some embodiments, however, less than 1° A caffeine is present, less than 0.5% is present, or the composition can be substantially caffeine free.

Other xanthines are also believed or known to have vasoconstrictive effects relative to the skin or other potentially useful effects relative to antiperspirant and deodorant products, including pharmacological effects related to those of caffeine, and thus xanthines such as methylxanthines and derivatives thereof are considered within the scope of certain embodiments of the present invention. Methylxanthines include theophylline (1,3-dimethyl-7H-purine-2,6-dione, also known as dimethylxanthine), caffeine (1,3,7-trimethyl-1H-purine-2,6(3H,7H)-dione, also known as 1,3,7-trimethylxanthine or methyltheobromine), and theobromine (3,7-dimethyl-2,3,6,7-tetrahydro-1H-purine-2,6-dione, also known as 3,7-dimethylxanthine or xantheose). Derivatives of xanthine compounds including salts thereof may be used, including caffeine citrate and other salts with carboxylic acids.

Xanthine-containing herbs include Camellia sinensis (Tea), Coffea arabica (Coffee beans), Cola nitida (Kola), Cola acuminata, Theobroma cacao (Cacao), Theobroma bicolor, Theobroma angustifolium, Ilex paraguariensis (Mate), Pauffinia cupana (Guarana), Banisteriopsis inebrians, DaviIla rugosa, Euonymus europaeus, Erodium spp., Genipa spp., Lippia multiflora, Maytenus spp., Sterculia spp., Tylophora moffissima, the Yaupon Holly (Ilex vomitoria) and Villarsia spp. The xanthine compound may be provided via a plant extract or mixture of plant extracts. In one embodiment, the composition comprises an herbal extract which has been substantially enriched in xanthine content beyond that available by forming an extract with heated water or ethanol alone. In one embodiment, for example, substantially pure caffeine and a xanthine-containing plant extract are combined to form a personal care composition with deodorant and/or antiperspirant properties.

The mass ratio of the carboxylic acid to total xanthine compounds may be any practical finite number such as from 0.1 to 5.0, from 0.5 to 30, from 1 to 10, or from 1 to 5, or substantially greater than 1 such as about 1.5 or greater.

The mandelic acid in the final composition may have a concentration of about 0.2% to 10%, from 0.5% to 8%, from 0.5% to 5%, and from 1% to 8%.

When a portion of the finished composition such as a pellet of mass 0.2 g is combined with twice the mass of liquid water and mixed or rubbed together at room temperature and placed in contact with a pH paper to estimate the pH, the apparent pH may be from 2 to 6, such as from 2.8 to 5.7, from 3 to 5.5, from 3.2 to 5.5, and from 3.5 to 5.4. A suitable pH paper is the Hydrion® pH 3.0-5.5 paper, as well as Hydrion® pH 0.0 to 6.0 paper.

Without wishing to be bound by theory, the role of mandelic acid or other suitable carboxylic acids is believed to be that of an acidifying composition that reduces the pH of the skin to a level that limits the growth of the bacteria that produce undesirable odors. Such bacteria can include Corynebacteria and Propionobacteria that dwell on the skin. In addition or alternatively, the reduced pH creates an environment that protects or maintains healthy microbial flora on the skin, thereby controlling the levels of less desirable bacteria that may produce unwanted odors.

### Definitions

As used herein, “deodorants” and “antiperspirants” both refer to compositions that are effective in directly or indirectly reducing unwanted body odors associated with perspiration and/or bacteria on the surface of the skin. “Deodorants” may reduce odor through a variety of means, and such means in the various embodiments of the present invention may include suppression of bacterial activity, antimicrobial mechanisms, chemical interference with odor generation mechanisms, removal or modification of feedstuff for odor-producing bacteria, and the like. “Antiperspirants” generally work to reduce the production of perspiration, and may do so, for example, by decreasing the size of pores associated with sweat glands, blocking sweat glands, reducing the flow of blood or other fluids to sweat glands, and the like. A deodorant may function as an antiperspirant but need not do so to be a deodorant. The compositions of various embodiments of the present invention may generally be described as deodorants and in many cases may generally be described as antiperspirants, though a composition that has limited antiperspirant efficacy is not necessarily outside the scope of the claimed invention, which is defined by the claims appended hereafter.

As used herein, a “solvent” for mandelic acid includes water, aqueous solutions, or a variety of other suitable compounds alone or in combination with water or other solvents described herein, such as ethanol, propanol, butanol, 1-3 propanediol, 1-2 propanediol (also known as propylene glycol), 3-phenyl-1-propanol, (2,2-Dimethyl-1,3-dioxolan-4-yl)methanol (also known as Solketal, isopropylidene glycerol, or Augeo Multi Clean), glycerin, pentylene glycol, 2-methoxy-2-phenylethanol, and 2-phenylethanol.

As used herein, a “thickener” is an agent that can substantially increase the viscosity of a solution of mandelic acid in a solvent. Thickeners may include starches such as native starches, modified starches, cold-water soluble starches, and the like, including but not limited to corn starch, tapioca starch, potato starch, cassava starch, arrowroot starch, wheat flour, sago, cationic starches such as cationic corn starch, etc. Thickeners may also include gums such as xanthan gum, guar gum, Sclerotium gum, locust bean gum, acacia gum, konjac gels, alginin and its derivatives, namely, alginic acid, sodium alginate, potassium alginate, ammonium alginate, and calcium alginate, and the like. Polysaccharide gums and other polysaccharide thickeners may be used such as pullulan, pectin, agar, gelatin, and carrageenan (both kappa and iota forms) can be considered. Cellulose derivatives may also be used such as cellulose ether derivatives such as carboxymethyl cellulose, methyl cellulose, ethyl cellulose, ethylmethyl cellulose, hydroxyethyl cellulose, hydroxymethyl cellulose, hydroxyethylmethyl cellulose, hydroxypropyl cellulose, hydroxypropyl methyl cellulose, hydroxypropyl ethyl cellulose, hydroxyalkylcellulose polymers, and the like. Mineral agents may be used such as slurries of clay materials such as kaolin, thickening waxes sold for cosmetic purposes, betonite, laponite, attapulgite, montmorillonite, hydroxyapatite, talc, etc. Various polymers may also be used such as polyvinyl alcohol, polylactic acid, carboxomer polymers, various superabsorbent polymers, and the like. For embodiments with silicone compounds, silicone resins, dimethicones, and various siloxanes may be considered such as cyclopentasiloxane.

As used herein, a “semi-solid” refers to a combination of solid and liquid materials or a composite material with multiple phases or discrete components which does not readily flow under the force of gravity when a unit such as a 5-cm cube of the material rests on a flat surface at 20° C., but which can deform and flow under shear. When measured at a shear rate of 0.5 sec⁻¹, the viscosity may be at least 15,000 centistokes, such as from 15,000 to 10,000,000 centistokes, from 30,000 to 10,000,000 centistokes, from 50,000 to 5,000,000 centistokes, or from 80,000 to 5,000,000 centistokes. Commercial deodorant sticks, whether based on waxy material, aqueous gels, or silicone compounds, are commonly semi-solids. Viscosity can be measured with a Brookfield viscometer.

As used herein, “emulsifying wax” refers to waxy materials that promote emulsification of an aqueous or polar phase with an oily, non-polar phase. Emulsifying waxes generally contain compounds derived from fatty acids such as fatty alcohols, esters, and other materials such a Polysorbate 60 or other emulsifiers. Emulsifying waxes may be plant derived, such as derivatives of palm oil, soy oil or olive oil. Examples include the combination of cetearyl alcohol and glyceryl stearate, such as Ritamulse SCG (sometimes known as Emulsimulse) from Rita Corp. (Crystal Lake, Illinois), which is a combination of glyceryl stearate, cetearyl alcohol, and sodium stearoyl lactylate; Emulsifying Wax NF (cetostearyl alcohol and polysorbate 60), and Polawax (cetearyl alcohol, PEG-150 stearate, polysorbate 60, and steareth-20). A useful plant-derived example is Milliard® All Natural Emulsifying Wax (Milliard Brands, Lakewood, N.J.) said to be derived from palm oil, with the INCI name of cetostearyl alcohol and polysorbate 80. Another natural emulsifying wax derived from olive oil is the combination of cetearyl olivate and sorbitan olivate, marked at Olivem 1000® by Hallstar Beauty (Chicago, Ill.). Emulsifying waxes are generally taught to be used at a level of 2 to 5% of the mass of the emulsion being made (e.g., the combination of the oil and aqueous phases). In some cases, we have found higher levels are useful, as 5% or greater, 6% to 23%, 7% to 17%, or 7% to 15%, and 8% to 14% of the combined mass of the oil and solvent phase prior to the addition of further solids such as starch.

As used herein, the “waxy phase” consists of the non-aqueous or non-polar materials that are combined as part of the process for forming the deodorant stick described herein. It may comprise waxes, oils, fatty acids, and related oleophilic materials. Thus, the waxy phase may comprise:

waxes such as beeswax, white beeswax, candelilla wax, carnauba wax, microcrystalline wax, paraffin wax, hydrogenated castor oil, rice bran wax, berry wax, myrica fruit wax, laurel wax, castor wax (hydrogenated castor oil), sunflower wax, rose wax, orange wax, momosa wax, jasmine wax, and so forth;

alcohols such as stearyl alcohol, behenyl alcohol, cetyl alcohol, cetearyl alcohol, isostearyl alcohol, lauryl alcohol, oleyl alcohol, caprylic alcohol, myristyl alcohol, polyols such as glycerin, glycols such as pentylene glycol, and the like;

fatty esters such as isopropyl myristate, isopropyl palmitate: Used in cosmetics both as a thickening agent and emollient, glyceryl stearate, tridecyl trimellitate, jojoba esters, pentaerythrityl tetraisostearate and other pentaerythrityl tetraesters, lauryl laurate, cetyl esters, PEG stearate, etc.;

oils such as Helianthus annus (sunflower) seed oil, soybean oil, almond oil, walnut oil, coconut oil, caprylic/capric triglicerides, MCT oil, jojoba oil, castor oil, etc.;

fatty acids such as stearic acid, palmitic acid, lauric acid, oleic acid, myristic acid, palmitoleic acid, etc.;

butters such as shea butter, Mangifera indica (mango) seed butter, etc.; and

soaps (metal salts of fatty acids) such as zinc stearate, magnesium stearate, sodium oleate, etc.

### BRIEF DESCRIPTION OF THE DRAWINGS

FIG. 1 depicts a stick of deodorant in a dispenser according to certain embodiments of the present invention.

### DETAILED DESCRIPTION

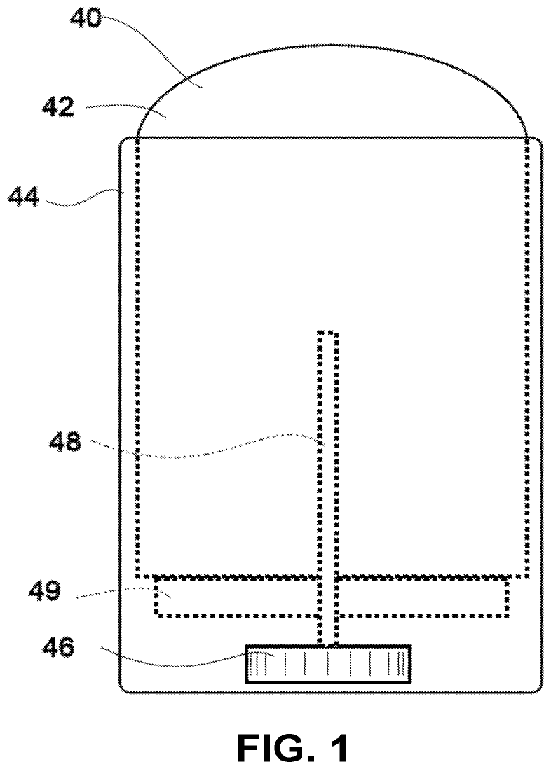

FIG. 1 depicts a stick dispenser 40 comprising a stick of a deodorant composition 42 contained within the walls of a container 44 having a turnable knob 46 attached to a threaded spindle 48 that drives the motion of an associated platform 49 on which the stick of a deodorant composition 42 rests. Thus, by turning the knob 46, the amount of the stick of a deodorant composition 42 rising above the walls of a container 44 can be adjusted. A cap for the container 44 is not shown. Such dispensers may be filled by pouring a slurry or melt into the dispenser and allowing the slurry to harden, or may be formed by packing solid or semi-solid material into the dispenser and then compressing it and/or heating it to form a suitable deodorant stick.

Further Details

Stick deodorants and related cosmetic stick products are known in a variety of forms, all of which may be suitably adapted for the various embodiments described herein. For example, cosmetic sticks having antiperspirant and/or deodorizing effects and based on alcoholic soap gels and/or propylene glycol soap gels are well known (e.g., see U.S. Pat. Nos. 2,900,306, “Cosmetic and Deodorant Sticks,” issued to Slater, Aug. 1959; 2,857,315, “Propylene Glycol Soap Gel Stick Anti-Perspirant,” issued Oct. 1958 to Teller; and U.S. Pat. No. 2,933,433, “Stable Anti-Perspirant Stick,” issued April 1960 to Teller et al.). A translucent stick product is described in U.S. Pat. No. 5,650,143, “Deodorant Cosmetic Stick Composition,” issued Jul. 22, 1997 to Bergmann.

### Examples

The ingredients mentioned in the Examples below were drawn from the following, unless otherwise specified:
almond oil:: Formulator Sample Shop, Iron Station, NC
arrowroot starch: Bob's Red Mill, Milwaukie, Oregon
beeswax: Sky Organics, Delray Beach, FL
cacao butter: Cacao Butter Wafers, Terrasoul Foods, Fort Worth, Tex.
caffeine: 200 mg capsules, Bulk Suplements, Henderson, NV
candelilla wax: TKB Trading, Oakland, Calif.
caprylic capric triglycerides MCT: Formulator Sample Shop, Iron Station, NC
cetyl alcohol: Formulator Sample Shop, Iron Station, NC
coconut oil: LouAna Coconut Oil, Ventura Foods, Brea, Calif.
emulsifying wax: Milliard® Emulsifying Wax, Milliard Brands, Lakewood, N.J.
laponite powder: Laponite XL21, BYK USA, Inc., Gonzales, TX
lauryl laurate: TKB Trading, Oakland, Calif.
Lip Stick Base: TKB Trading, Oakland, Calif. (a mixture of castor seed oil, cetyl stearyl alcohol, olive fruit oil, beeswax, hydrogenated castor oil, glycine soybean lipids, lauryl laurate, carnauba wax, candelilla wax)
mandelic acid powder: Pure Health Botanicals, St. Charles, Ill.
palmitic acid 98%, Acme-Hardesty, Blue Bell, PA
shea butter (unrefined): TKB Trading, Oakland, Calif.
Softisan: FSS Softisan 378, Formulator Sample Shop, Iron Station, NC (this material is a lanolin-like material made from a blend of triglycerides based on saturated even-numbered, unbranched natural fatty acids of vegetable origin)
stearyl alcohol: Alcohol 1989 N F Pastilles, Acme-Hardesty, Blue Bell, PA
tapioca starch: Erawan Marketing Co., Bangkok, Thailand
TKB gelmaker CC: TKB Trading, Oakland, Calif. (this material is a blend of dicaprylyl carbonate, stearalkonium hectorite and propylene carbonate, and is used to create gels)
water is distilled water unless otherwise specified
xanthan gum: Carrington Farms, Closter, NJ

Example 1 was prepared without an added thickener from a relatively volatile solvent system. 0.398 g of mandelic acid powder was placed in a beaker with 4.11 g of 40% ethanol in water and 0.62 g of glycerin. The mandelic acid was then stirred to dissolve it completely in the solvent system. To this was added 2.60 g of cacao butter, 2.85 g of beeswax, 1.16 g of coconut oil, 1.25 g of caprylic capric triglycerides MCT, and 1.90 g of stearyl alcohol. This beaker was placed in a bath of hot water at about 80° C. After stirring, the aqueous phase was not mixing well with the oil phase, so 0.86 g of emulsifying wax was added and blended in, resulting in a good emulsion. The combination remained above 60° C. for about 5 minutes. As the combination cooled below 60° C., 3.0 g of tapioca starch was rapidly blended in with a whisk and the mixture was poured into a deodorant mold and allowed to cool. The resulting solidified material was a somewhat waxy solid with a smooth and pleasant texture having no hint of graininess. Rubbing a slight amount onto the fingers and then adding a drop of water, and touching that water to a pH strip indicated a pH around 3.

Example 2. An acid-starch paste was made by combining 17.5 g of mandelic acid with 50 ml of water and heating to dissolve. After heating and stirring, about 5 g of water were lost. This was Acid Solution 1.3.11 g of tapioca starch were then combined with 3.1 ml of water to form a uniform slurry at about 22° C. Then 25.0 g of Acid Solution 1 was blended into the starch in a beaker, a stirring rod was added and the mixture was placed on a magnetic stirrer hot plate and gradually heated and stirred. As the mixture rose above about 50° C., it began to form a thickened paste. This paste is Acid Paste 1.

Then 0.38 g palmitic acid was combined with 5.00 g lipstick base, 1.03 g lauryl laurate, 3.00 g coconut oil, 4.60 g stearyl alcohol, 2.43 g cacao butter, and 200 mg caffeine. This was melted in a double boiler. Then 1.26 g of Acid Paste 1 was blended in with a whisk, and as the mixture cooled to about 53° C., 4.5 g of arrowroot starch was blended in. The mixture was then spooned into a 15 ml oval deodorant stick/lip balm mold and allowed to solidify. The starch concentration seemed somewhat too high for this mixture, with some evidence that portions of the acidic paste were not fully dispersed by hand stirring with a whisk.

Example 3. An acidic paste was made from 1.351 g laponite powder and 0.226 g xanthan gum with 25.7 g of Acid Solution 1. This was also stirred and heated on a magnetic stirrer plate until it reach a temperature of 45° C. This was Acid Paste 2, 1.00 g of which was stirred into an oil phase formed by heating the following on a double boiler to about 75° C.: 200 mg of caffeine combined with 0.60 g shea butter, 7.00 g lipstick base, 4.50 g stearyl alcohol, 1.13 g beeswax, 1.48 g cacao butter, 0.51 g candelilla wax, and 1.03 g of almond oil. After Acid Paste 2 was blended with the oil phase using a whisk, 3.00 g of arrowroot starch were blended in and the mixture was spooned into a 15 ml deodorant/lip balm container and cooled. The white, opaque solid had a very firm texture and may have had slightly too much starch for easy dispensing onto the skin, or the laponite and gum combination may have increased the viscosity of the stick relative to some other mixtures with starch. When a small quantity was blended with water and placed on Hydrion® 3.0 to 6.0 pH paper, a pH of about 4.0 was indicated.

Example 4. Acid Paste 3 was prepared by diluting Acid Paste 1 with about 15% added water to reduce viscosity and improve blending. An oil phase was prepared using 1.50 g Softisan, 2.50 g caprylic capric triglycerides MCT, 2.10 g stearyl alcohol, 2.65 g lipstick base, 5.00 g coconut oil, 0.79 g emulsifying wax, and 200 mg caffeine. The oil phase, heated in a double boiler at about 75° C., was blended with 0.92 g of Acid Paste 3 and then after about several minutes of stirring and gradual cooling, 2.13 g of arrowroot starch was blended in at about 55° C. A smooth, slick, pleasant-feeling solid was obtained after cooling that readily dispenses against the skin, with no hint of graininess. The effective pH was estimated at about 4.4 to 4.8. One problem, though, is that some clear zones apparently from Acid Paste 3 remained at the bottom of the small container used to mix the composition, suggesting that mixing was inadequate and perhaps emulsifying wax would have helped. The pH may have been lower had all the starch blended in more completely.

Example 4. An oil phase was prepared using 7.00 g caprylic capric triglycerides MCT, 3.5 g stearyl alcohol, 2.00 g cacao butter, 2.21 g coconut oil, 2.5 g emulsifying wax, 1.00 g shea butter, and 200 mg caffeine. This was combined at 74° C. and then 2.12 g of Acid Paste 3 was blended in. Following slight cooling, 3.85 g of arrowroot starch was blended in. The resulting solid had a firm, smooth texture that could dispense well against the skin. Each of 3 different pH papers (0 to 14, 0 to 6, and 3.0 to 5.5) suggested the effective pH after being rubbed with water was near 3.0.

Example 5. An oil phase was prepared by combining 4.00 g of caprylic capric triglycerides MCT, 5.00 g cetyl alcohol, 3.50 g shea butter, 0.50 g cacao butter, 1.05 g coconut oil, and 200 mg caffeine. It was mixed at 74° C. 2.00 g of Acid Paste 3 was blended in and then, after slight cooling, 2.70 g of arrowroot starch was added. This run showed some slight separation in the bottom of the container, suggesting emulsifying wax or more intense mixing may have been helpful.

Example 6. Acid Paste 4 was made by combining 13 g mandelic acid in 100 ml of water with 8.35 g arrowroot starch and cooking in a frying pan to create a translucent paste. Then 9.0 g almond oil was combined with 4.26 g stearyl alcohol, 2.68 g cacao butter, 0.200 g caffeine and 1.57 g emulsifying wax. After melting, 2.15 g of Acid Paste 4 was blended in, and then, after slight cooling, 3.68 g of arrowroot starch was blended in. A portion of the hot composition was poured into a 15 ml deodorant/lip balm container. The cooled result had a smooth tactile feel but was also on the firm side.

Example 7.7.36 g of the remaining composition from Example 6 was combined with 0.84 g cacao butter, 0.79 g beeswax, 0.34 g shea butter, and 1.37 g arrowroot starch. The resulting mix had a pH of about 5.5. This was melted again and 1.25 g of Acid Paste 4 was blended in to lower the pH.

Example 8. An oil phase was made from 8.9 g caprylic capric triglycerides MCT, 0.91 g cacao butter, 1.00 g shea butter, 2.47 g beeswax, 5.25 g stearyl alcohol, 3.00g emulsifying wax, 200 mg caffeine, and 1.10 g almond oil. After melting, 4.0 g of Acid Paste 4 was blended in, and then 6.2 g of arrowroot starch was added. The result was too starchy and lacked the strength expected in a waxy composition. A portion of the result, 13.4 g, was melted and combined with 2.1 g stearyl alcohol, 1.15 g almond oil, and 1.35 g candelilla wax. The pH was tested and was on the high side, so the mix was remelted and 4.9 g of Acid Paste 4 was blended in. The result had a more acceptable feel and gave an effective pH when wetted of about 3-3.5.

A wide variety of other examples were also carried out. Initialy demonstration of the mandelic acid stick concept was made by blending combinations of corn starch and mandelic acid to form a viscous paste with mandelic acid concentrations from 6% to 15%, using starch concentrations from about 4% to 7%. This was then blended with various oil phases, including combinations with emulsifying wax, beeswax, and other waxes, with added TKB gelmaker CC in some cases. Then, rather than stirring in starch or other solids, the mix of the oil phase and acid paste was blended in with roughly an equal part of molten commercial deodorant or antiperspirant. Some of the best results in this early phase were obtained with aluminum-free deodorants with a waxy base in which caprylic capric triglycerides were the lead ingredient, such as Women Mitchum®'s Natural Power Bamboo Powder deodorant, comprising caprylic capric triglycerides, corn starch, coconut oil, stearyl alcohol, tapioca starch, arrowroot starch, sodium bicarbonate, and other ingredients. When the acid paste was combined with the alkaline Mitchum® deodorant, the bicarbonate reacted with the mandelic acid releasing small bubbles of carbon dioxide that caused the molten mixture to foam up, changing its texture and appearance significantly, but after cooling, good results were obtained with a firm texture and, due to excess acid, a pH in the range of 3 to 4. More challenging were attempts using Native® natural deodorant and other products with larger amounts of sodium bicarbonate or in which alkaline magnesium hydroxide was the lead ingredient. Acidic mixes could be made with the starch-acid method, but the final results in some cases were not satisfying in their texture. Better results were obtained by making the composition from scratch, rather than seeking to turn non-acidic compositions into an acidic mixture.

Among the efforts to combine an acidified waxy phase with an existing deodorant stick, a silicone-based antiperspirant was used, Degree® brand from Unilever, with cyclopentasiloxane as the primary ingredient. This was melted down and combined with 1.48 g of Acid Paste 4, giving an acidic mix. However, the volatile nature of the cyclopentasiloxane resulted in mass loss from the material. But the final result, after resolidification, did not show obvious signs of failure.

### REMARKS

When introducing elements of aspects of the invention or the embodiments thereof, the articles “a,” “an,” “the,” and “said” are intended to mean that there are one or more of the elements, and thus may include plural referents unless the context clearly dictates otherwise. The terms “comprising,” “including,” and “having” are intended to be inclusive and mean that there may be additional elements other than the listed elements.

Having described aspects of the invention in detail, it will be apparent that modifications and variations are possible without departing from the scope of aspects of the invention as defined in the appended claims. As various changes could be made in the above compositions, products, and methods without departing from the scope of aspects of the invention, it is intended that all matter contained in the above description shall be interpreted as illustrative and not in a limiting sense.

While the foregoing description makes reference to particular illustrative embodiments, these examples should not be construed as limitations. The inventive system, methods, and products can be adapted for other uses or provided in other forms not explicitly listed above, and can be modified in numerous ways within the spirit of the present disclosure. Thus, the present invention is not limited to the disclosed embodiments, but is to be accorded the widest scope consistent with the claims below.

We claim:

1. An acidic stick deodorant comprising a personal care composition comprising a solid or semi-solid waxy phase comprising one or more waxes, a starch, and mandelic acid associated with a thickener substantially uniformly dispersed in the waxy phase.

2. The acidic stick of claim 1, wherein the thickener is selected from one of a starch, a swellable mineral, and a gum.

3. The acidic stick of claim 1, having a viscosity at 25° C. of at least 100,000 cps, an effective pH between 2 and 5.5.

4. A method for preparing an acidic stick deodorant comprising:1) preparing a thickened aqueous phase comprising at least 3% mandelic acid and a thickener in an aqueous solution; 2) melting one or more waxes and at least one of an oil, a fatty acid, a fatty ester, a fatty alcohol, a soap, and a butter to form a molten waxy phase, 3) combining the thickened aqueous phase with the waxy phase, 4) combining a powder with the combination of the aqueous phase with the waxy phase.

5. The method of claim 4, having an effective pH between 2 and 6.

6. The method of claim 4, having an effective pH between 3 and 5.5.

7. The method of claim 4, further comprising combining caffeine with at least one of the aqueous phase and the oil phase.
